(19) United States
(12) Patent Application Publication
(10) Pub. No.: US 2009/0069417 A1
(43) Pub. Date: Mar. 12, 2009

(54) CAROTENOID OXIDATION PRODUCTS AS CHEMOPREVENTIVE AND CHEMOTHERAPEUTIC AGENTS

(76) Inventors: Yoav Sharoni, Omer (IL); Joseph Levy, Omer (IL); Yoram Sela, Ra'anana (IL); Zohar Nir, Meitar (IL)

Correspondence Address: NIXON & VANDERHYE, PC, 901 NORTH GLEBE ROAD, 11TH FLOOR, ARLINGTON, VA, 22203, US

(21) Appl. No.: 12/090,174

(22) PCT Filed: Oct. 5, 2006
(86) PCT No.: PCT/IL06/01169

§ 371 Date: Oct. 22, 2008

(30) Foreign Application Priority Data
Oct. 11, 2005 (IL) 171374

(51) Int. Cl. A61K 31/343; A61K 31/01; A61K 31/045; A61K 31/11; A61P 35/00; A61K 31/12; A61K 31/336

(52) U.S. Cl. 514/469; 514/762; 514/739; 514/475; 514/675; 514/693

(57) ABSTRACT

The present invention relates to a method for the chemoprevention and treatment of cancer, by administering a pharmaceutical composition comprising a carotenoid derivative, such as a derivative of lycopene, a- and b-carotene, phytoene, phytofluene, lutein, zeaxanthin, α- and β-cryptoxanthin, canthaxanthin, astaxanthin, or other carotenoid. The carotenoid derivative is a carotenoid oxidation product, and is preferably an aldehyde derivative, a dialdehyde derivative or a ketone derivative. The carotenoid derivative can be a derivative of any naturally occurring carotenoid, such as those found in tomatoes and other fruits and vegetables.

### FIELD OF THE INVENTION

The present invention relates to a method for the chemoprevention and treatment of cancer, by administering a pharmaceutical composition comprising a carotenoid oxidation product, e.g., an oxidation product of lycopene, α- and β-carotene, phytoene, phytofluene, lutein, zeaxanthin, α- and β-cryptoxanthin, canthaxanthin, astaxanthin or any other carotenoid. The carotenoid oxidation product can be a derivative of any naturally occurring carotenoid, e.g., carotenoids found in tomatoes and other fruits and vegetables.

### BACKGROUND OF THE INVENTION

There is considerable epidemiologic evidence suggesting an association between the consumption of fruits and vegetables and reduced incidence of cancer. In particular, carotenoids and other plant constituents have been implicated as cancer-preventive agents (1). β-Carotene has received the most attention because of its provitamin A activity and its prevalence in many foods. However, findings from intervention studies with β-carotene were disappointing, and thus other carotenoids such as lycopene, the main tomato carotenoid, became the subject of more intensive investigation. A comprehensive analysis of the epidemiologic literature on the relation of tomato consumption and cancer prevention has been published by Giovannucci (2), who found that most of the reviewed studies reported an inverse association between tomato intake or blood lycopene level and the risk of various types of cancer. Giovannucci suggested that lycopene may contribute to these beneficial effects of tomato-containing foods but that the anticancer properties could also be explained by interactions among multiple components found in tomatoes such as phytoene, phytofluene, and β-carotene. The applicants of the present invention have shown that lycopene inhibits mammary, endometrial, lung and leukemic cancer cell growth in a dose-dependent manner (IC50 ca. 2 μmol/L; U.S. Pat. No. 5,827,900).

The biochemical mechanisms involved in the chemoprotective effects of fruits and vegetables in general and of tomatoes in particular are not completely understood. In recent years, evidence has accumulated indicating that the beneficial action is due, at least in part, to the induction of phase II detoxification enzymes (3). These enzymes detoxify many harmful substances by converting them to hydrophilic metabolites that can be excreted readily from the body. Phase II enzymes, such as NAD(P)H: quinone oxidoreductase (NQO1) and γ-glutamylcysteine synthetase (GCS) are inducible in animals and humans, and a strong inverse relationship exists between their tissue levels and susceptibility to chemical carcinogenesis.

The coordinated induction of phase II enzymes is mediated through cis-regulatory DNA sequences located in the promoter or enhancer region, which are known as antioxidant responsive elements (ARE). Stimulation of the ARE transcription system is an established mechanism for the mobilization of the body's defense system against carcinogens and other harmful compounds. The major ARE activating transcription factor Nrf2 (nuclear factor E₂-related factor 2) plays a central role in the induction of antioxidant and detoxifying genes. Under basal conditions, Nrf2 is located in the cytoplasm and is bound to an inhibitory protein, Keap1. Upon challenge with inducing agents, it is released from Keap1 and translocates to the nucleus. It has recently been shown by the applicants of the present invention that in transiently transfected cancer cells, lycopene is capable of transactivating the expression of reporter genes fused with ARE sequences (4). A mixture of two other tomato carotenoids, phytoene and phytofluene, was also effective in activation of ARE. By activating this system, tomato carotenoids induce the production of phase II detoxification enzymes.

Although carotenoids per se are thought to have biological effects, they are susceptible to oxidation under certain conditions to produce a number of compounds. Retinal and β-apo-cartoenals with different carbon chain length have been known to be formed from β-carotene by non-enzymatic oxidation under various conditions such as auto-oxidation in solvents, oxidation with peroxy radical initiators, singlet oxygen, cigarette smoke, and co-oxidation by lipoxygenase. Retinoic acid was also suggested to form by autoxidation of β-carotene in benzene. Peroxy radical oxidation products of β-carotene are also known, as well as the effects of ozone and oxygen on the degradation of carotenoids in an aqueous model system. Carotenoids and their oxidation products have also been extracted from human plasma.

Few studies have investigated the metabolism of lycopene in biological systems, and very little is known about oxidative break-down products of lycopene in humans. The first report of a metabolite in human plasma was that of 5,6-dihydroxy-5′,6′-dihydrolycopene resulting from oxidation of lycopene (5). It has also been reported that 2,6-cyclolycopene-1,5-diol A and B are in vivo oxidative metabolites of lycopene in humans (6, 7). Ferreira et al. used post-mitochondrial fractions of rat intestinal mucosa to identify two types of lycopene metabolites: a) cleavage products (3-keto-apo-13-lycopenone and 3,4-dehydro-5,6-dihydro-15,15′-apo-lycopenal); and b) oxidation products (2-apo-5,8-lycopenal-furanoxide; lycopene-5,6,5′,6′-diepoxide, lycopene-5,8-furanoxide isomer (I), lycopene-5,8-furanoxide isomer (II), and 3-keto-lycopene-5′,8′-furanoxide) (8). Kim et al. reported a homologous series of carbonyl cleavage products of variable chain lengths formed by auto-oxidation of lycopene in vitro, and suggested that lycopene might be cleaved to a series of apolycopenals and short-chain carbonyl compounds under oxidative conditions in biological tissues (9). Caris-Veryat et al. reported the formation of various aldehyde and dialdehyde degradation products resulting from oxidation of lycopene with potassium permanganate and metalloporphyrin-catalyzed atmospheric oxygen (10).

Other tomato carotenoids such as phytoene and phytofluene can be oxidized in a similar manner giving rise to similar oxidation products. Phytoene and phytofluene are lycopene precursors which are structurally similar to lycopene, with the exception that these molecules contain fewer conjugated double bonds. Oxidation of these compounds should give rise to hydrogenated analogues of the oxidation products of lycopene (for example cleavage of the central bond). Araki et al developed synthetic acyclic analogs of retinoic acid, which according to their structure can be putative derivatives of phytoene and phytofluene (11).

Several studies have linked lycopene oxidation products to cancer chemoprevention and treatment. Aust et al. reported that the dialdehyde 2,7,11-trimethyl-tetradecahexaene-1,14-dial, formed by in vitro oxidation of lycopene with hydrogen peroxide/osmium tetroxide, stimulates gap junctional communication (GJC) in WB-F344 rat liver epithelial cells. Stimulation of GJC between cells is thought to be one of the protective mechanisms related to the cancer-preventive activities of carotenoids (12). Zhang et al. reported that (E,E,E)-4-methyl-8-oxo-2,4,6-nonatrienal, a cleavage product formed by auto-oxidation of lycopene, induces apoptosis in HL-60 human promyelocytic leukemia cells (13). Similar effects were observed for the acyclic carotenoids phytoene, phytofluene and ξ-carotene, also present in tomatoes, as well as for oxidation mixtures of lycopene (14).

New innovative approaches are urgently needed at both the basic science and clinical levels to develop compounds which are useful for the chemoprevention and treatment of cancer.

### SUMMARY OF THE INVENTION

The present invention relates to a method for the chemoprevention and treatment of cancer, by administering a pharmaceutical composition comprising a carotenoid derivative e.g., a derivative of lycopene, α- and β-carotene, phytoene, phytofluene, lutein, zeaxanthin, α- and β-cryptoxanthin, canthaxanthin, astaxanthin or any other carotenoid. The carotenoid derivative is a carotenoid oxidation product, and is preferably an aldehyde derivative, a dialdehyde derivative or a ketone derivative. The carotenoid derivative can be a derivative of any naturally occurring carotenoid, e.g., carotenoids found in tomatoes and other fruits and vegetables.

As demonstrated herein, the applicants of the present invention tested the anti-cancer activity of compounds that have the structure of the putative oxidative products of lycopene and other carotenoids. The carotenoid derivatives have anticancer activity, as demonstrated by their ability to induce an antioxidant response element (ARE) and inhibit cancer cell proliferation. Surprisingly, several of these derivatives were found to be more active than the parent carotenoid.

Thus, in one embodiment, the present invention provides a method of preventing the onset of cancer in a subject, comprising the step of administering to the subject a pharmaceutical composition comprising a carotenoid oxidation product (e.g., an oxidation product of lycopene, α- and β-carotene, phytoene, phytofluene, lutein, zeaxanthin, α- and β-cryptoxanthin, canthaxanthin, astaxanthin or any other carotenoid), in an amount effective prevent the onset of cancer in the subject.

In another embodiment, the present invention provides a method of inhibiting cancer cell proliferation in a subject, comprising the step of administering to the subject a pharmaceutical composition comprising a carotenoid oxidation product, in an amount effective to inhibit cancer cell proliferation in the subject.

In yet another embodiment, the present invention provides a method of delaying the progression of cancer in a subject, comprising the step of administering to the subject a pharmaceutical composition comprising a carotenoid oxidation product, in an amount effective to delay the progression of cancer in the subject.

In yet another embodiment, the present invention provides a method of treating cancer in a subject, comprising the step of administering to the subject a pharmaceutical composition comprising a carotenoid oxidation product, in an amount effective to treat cancer in the subject.

In a currently preferred embodiment, the carotenoid oxidation product is selected from the group consisting of dialdehyde derivatives (designated herein carotendials), aldehyde derivatives (designated herein carotenals) and ketone derivatives (designated herein carotenones). Other suitable oxidation products include but are not limited to epoxide derivatives, furanoxide derivatives, carboxylic acid derivatives, gamma-lactone derivatives, alpha-hydroxy ketone derivatives, diol derivatives, acetal derivatives, ketal derivatives, halogenated derivatives, acetylated derivatives and derivatives containing one or more alkynic bonds. Also contemplated are any one or more of these derivatives in which one or more of the double bonds has been reduced. Combinations of one or more of these functional groups in the oxidation products are also contemplated.

In another currently preferred embodiment, the carotenoid oxidation product is a lycopene oxidation product. A currently preferred lycopene oxidation product is a dialdehyde derivative of lycopene (designated herein diapo-carotendial or diapo-lycopendial) Examples of such dialdehyde lycopene oxidation products include but are not limited to diapo-8,8′-lycopendial (designated herein 8,8′), diapo-8′,12-lycopendial (designated herein 8′, 12), diapo-10,10′-lycopendial (designated herein 10,10′), diapo-12,12′-lycopendial (designated herein 12,12′), diapo-8′,15-lycopendial (designated herein 8′,15), and any combination thereof. In one embodiment, the carotenoid oxidation product is other than 2,7,11-trimethyl-tetradecahexaene-1,14-dial (also designated herein diapo-6,12′-lycopenedial).

The carotenoid oxidation products used in the present invention can be synthetic derivatives, prepared by any synthetic method known in the art. Further, the present invention also contemplates the use of oxidation products derived from naturally occurring carotenoids, e.g., by oxidation of naturally occurring carotenoids. The natural carotenoid can be any one or more of the carotenoids described herein, or any other putative carotenoid. The naturally occurring carotenoid, can be, for example carotenoids found in tomato products (e.g., tomatoes, tomato sauce, ketchup and the like), fermentation, watermelon, guava, grapefruit, and the like. In one embodiment, the carotenoid oxidation product is obtained by extracting the carotenoid from a natural source, followed by oxidation.

In another aspect, the present invention provides pharmaceutical compositions comprising a carotenoid oxidation product of the present invention, and a pharmaceutically acceptable carrier or excipient.

Without wishing to be bound by any particular mechanism or theory, it is contemplated that the carotenoid derivative induces the level and/or activity of antioxidant response element (ARE), which in turn induces the production of phase II enzymes, which are responsible for converting harmful carcinogenic compounds into less toxic compounds that are readily excreted by the body. As demonstrated herein, the carotenoid oxidation products are capable of inducing antioxidant response element (ARE) in a cell. In one embodiment, the carotenoid oxidation product increases the activity of ARE. Induction of ARE in turn induces phase II detoxification enzymes, which convert harmful carcinogenic compounds into less toxic compounds that are readily excreted by the body.

In one non-limiting embodiment, the carotenoid derivatives increase the activity of ARE by forming keap 1 adducts with the carotenoid derivative. This may cause in turn its dissociation from Nrf2 and the activation of the latter.

In some exemplary embodiments of the methods of the present invention, the carotenoid oxidation product is administered in an amount effective to induce an antioxidant response element (ARE) in the subject, thereby preventing the onset of cancer, inhibiting cancer cell proliferation, delaying the progression of cancer, and/or treating cancer in the subject.

The carotenoid oxidation products of the present invention have been shown to inhibit proliferation of several cancer cell lines. Therefore, the invention also relates to a method of inhibiting cancer cell proliferation, by contacting a cancer cell with a carotenoid oxidation product, in an amount effective to inhibit proliferation of the cancer cell. The methods of the present invention can be practiced in cells or tissue cultures, or in living organisms, for example humans. The carotenoid derivatives are potent against a wide variety of cancer cell lines, non-limiting examples of which include prostate cancer, liver cancer and breast cancer.

In one embodiment, the carotenoid oxidation products are more active as compared with the parent carotenoid. For example, the carotenoid oxidation product can be at least 10% more active as compared with the parent carotenoid, at least 50% more active, at least two fold more active, and the like. Further, the in-vivo activities of the oxidation products can differ from their in-vitro activities due to factors such as variability in oral bioavailabilities and dissolution of poorly water soluble compounds and other factors affecting biological activity. As such, the oxidation products may be more potent chemotherapeutic agents compared with the corresponding parent molecule.

The carotenoid oxidation products of the present invention are potent chemotherapeutic agents that are capable of inhibiting cancer cell proliferation in a wide variety of cancer cells. The present invention thus provides powerful methods to the chemoprevention and treatment of cancer that have not been previously described.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: ARE induction by lycopene and lycopene derivatives.

FIG. 2: ARE induction and inhibition of cell proliferation by lycopene and lycopene derivatives in human breast cancer cell lines. FIG. 2A: ARE induction in MCF-7 cells (left) and T47D cells (right); FIG. 2B: inhibition of cell proliferation in MCF-7 cells (left) and T47D cells (right).

FIG. 3: Inhibition of cell proliferation by lycopene and synthetic derivatives in a human prostate cancer cell line.

### DETAILED DESCRIPTION OF THE PRESENT INVENTION

The present invention provides powerful methods for the chemoprevention and treatment of cancer using carotenoid derivatives, e.g., derivatives of lycopene, α- and β-carotene, phytoene, phytofluene, lutein, zeaxanthin, α- and β-cryptoxanthin, canthaxanthin, astaxanthin, or any other carotenoid present in tomato extract. The derivatives are putative oxidation products of carotenoids, e.g., tomato carotenoids.

### Carotenoid Derivatives

The carotenoid derivatives of the present invention are putative oxidation products of carotenoids such as lycopene, α and β-carotene, phytoene, phytofluene, lutein, zeaxanthin, α and β-cryptoxanthin, canthaxanthin, astaxanthin, or any other carotenoid. The carotenoid oxidation products used in the present invention can be synthetic derivatives, prepared by any synthetic method known in the art. Further, the present invention also contemplates the use of oxidation products derived from naturally occurring carotenoids, e.g., by oxidation of naturally occurring carotenoids. The natural carotenoid can be any one or more of the carotenoids described herein, or any other putative carotenoid. The naturally occurring carotenoid, can be, for example carotenoids found in tomato products (e.g., tomatoes, tomato sauce, ketchup and the like), fermentation, watermelon, guava, grapefruit, and the like. In one embodiment, the carotenoid oxidation product is obtained by extracting the carotenoid from a natural source, followed by oxidation. The carotenoids can be extracted from the natural source using extraction techniques known to a person of skill in the art.

Carotenoid derivatives in which the carbon skeleton has been shortened by the formal removal of fragments from one or both ends of a carotenoid are referred to herein as ‘apo-carotenoids’ and ‘diapo-carotenoids’, respectively, as known in the art.

### A. Lycopene Derivatives

In a currently preferred embodiment, the carotenoid derivatives used in the methods of the present invention are putative oxidation products of the carotenoid lycopene. Any oxidation product of lycopene, whether synthetic or natural, can be used in the compositions and methods of the present invention.

In one embodiment, the lycopene derivatives are synthetically prepared by oxidizing lycopene, in accordance with any oxidation method known to a person of skill in the art. For example, the lycopene oxidation products can be formed by auto-oxidation of lycopene as described in Kim et al. (9) and Zhang et al (13), using hydrogen peroxide/osmium tetraoxide as described in Aust et al. (12), by ozonolysis of lycopene as described in Kim et al. (9) and in Nara et al. (14), by using potassium permanganate as described in Caris-Veyrat et al. (10), by using hydrogen peroxide and sulfuric acid as described in Yokota et al. (15), by using oxygen in the presence of a sensitizer (methylene blue) as described in Ukai et al. (16), or by any other synthetic oxidation method known to a person of skill in the art. The lycopene derivatives can also be obtained by enzymatic oxidation of lycopene as described in Ferreira et al. (8). The lycopene derivatives can also be prepared by isolation from, e.g., tomato and tomato products, for examples as described in Yokota et al. (15, 17). Lycopene and other carotenoid oxidation products can also be prepared by as described in United States published patent application US 2003/0158427.

The contents of all of the aforementioned references are incorporated by reference in their entirety as if fully set forth herein.

In addition, other methods of oxidation of alkenes to aldehydes, ketones and other oxidative products are known and could be applied to oxidation of lycopene and other carotenoids. For example alkenes can be oxidized to aldehydes or ketones in the presence of palladium chloride, water and air, usually with a co-oxidant as copper chloride. Another method for producing aldehydes is the hydroformylation of alkenes with carbon monoxide and hydrogen in the presence of a metallic catalyst.

It will also be apparent to a person of skill in the art, that in addition to being derived from lycopene, the lycopene derivatives of the present invention can be synthetically prepared from any other starting materials, using any conventional method known in the art.

Although the lycopene oxidation products of the present invention can be synthetically prepared, the present invention also contemplates the use of natural lycopene oxidation products formed in vivo, for example in the human body. Thus, in one embodiment, any one or more of the lycopene oxidation products described herein are putative oxidation products of lycopene that are formed in biological systems. Indeed, it has been suggested that oxidation products of lycopene and other carotenoids, rather than the intact carotenoid, are responsible for some of the biological activities of carotenoids, for example cancer prevention (9, 14). In fact, when the applicants of the present invention extracted a lycopene preparation with ethanol to separate the hydrophilic oxidized derivatives of this carotenoid from the parent molecule, the ethanolic extract transactivated the ARE reporter genes, in a dose-dependent manner, at a potency which was equivalent to that of the original lycopene. Since the concentration of the derivatives is clearly lower than that of lycopene, it has been suggested that one or some of the oxidized derivatives of lycopene is more active that the parent carotenoid.

A currently preferred lycopene oxidation product is a dialdehyde derivative (referred to herein interchangeably as diapo-carotendial or diapo-lycopendial). Such dialdehyde derivatives are described, for example, by Caris-Veyrat et al. (10), the contents of which are incorporated by reference in their entirety as if fully set forth herein. The dialdehyde derivatives are formed by cleavage of various bonds at the indicated carbon atoms of lycopene. Examples of these dialdehyde derivatives include but are not limited to: diapo-2,2′-lycopendial (2,2′); diapo-4,4′-lycopendial (4,4′); diapo-6,6′-lycopendial (6,6′); diapo-8,8′-lycopendial (8,8′); diapo-10,10′-lycopendial (10,10′); diapo-12,12′-lycopendial (12,12′); diapo-6,8′-lycopendial (6,8′) (also designated diapo-6′,8-lycopendial (6′,8)); diapo-6,10′-lycopendial (6,10′) (also designated diapo-6′,10-lycopendial (6′,10)); diapo-6,12′-lycopendial (6,12′) (also designated diapo-6′,12-lycopendial (6′,12)); diapo-8,10′-lycopendial (8,10′) (also designated diapo-8′,10-lycopendial, (8′, 10)); diapo-8,12′-lycopendial (8,12′) (also designated diapo-8′,12-lycopendial (8′,12)); diapo-8′,15-lycopendial (8′,15) (also designated diapo-8,15′-lycopendial (8,15′)); 2,4,6-octatrienedial, 2-(hydroxymethyl)-7-methyl-(E,E,E)-(9CI); 5-cis-4,4′-7,7′,8,8′,11,11′,12,12′,15,15′-decahydro-diapo-ψ,ψ-carotenedial (CAS No. 122440-45-3); 7,7′,8,8′,11,11′,12,12′,15,15′-decahydro-4,4′-diapo-ψ,ψ-carotenedial (CAS No. 122333-85-1); 7,7′,8,8′,11,11′,12,12′,15,15′-decahydro-6,6′-diapo-ψ,ψ-carotenedial (CAS No. 26906-73-0); and the like. Currently preferred examples of such dialdehyde derivatives include diapo-8,8′-lycopendial (8,8′), diapo-8′,12-lycopendial (8′,12), diapo-10,10′-lycopendial (10,10′), diapo-12,12′-lycopendial (12,12′), diapo-8′,15-lycopendial (8′,15), The structures of these derivatives are shown in Table 1 below.

TABLE 1
CHEMICAL STRUCTURE OF DIAPO-LYCOPENDIALS

| Name | Chemical Structure |
|---|---|
| Lycopene | 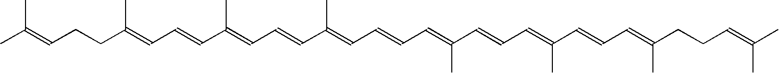 |
| diapo-2,2′-lycopendial (2,2′) | 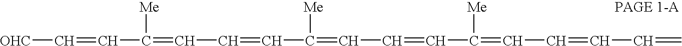 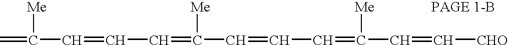 |
| diapo-4,4′-lycopendial (4,4′) | 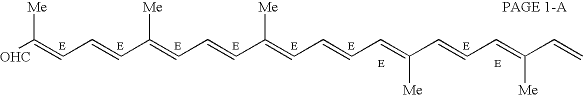 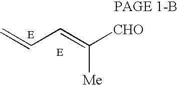 |
| diapo-6,6′-lycopendial (6,6′) | 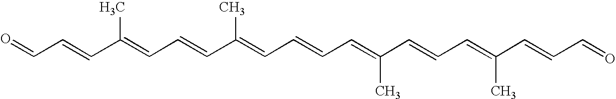 |
| diapo-8,8′-lycopendial (8,8′) | 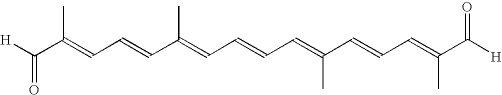 |
| diapo-10,10′-lycopendial (10,10′) | 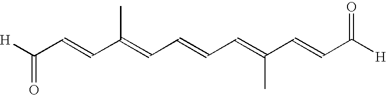 |
| diapo-12,12′-lycopendial (12,12′) | 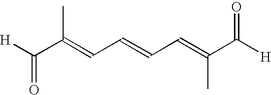 |
| Diapo-8,6′-lycopendial (8,6′) | 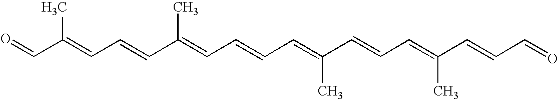 |
| diapo-6,10′-lycopendial (6,10′) | 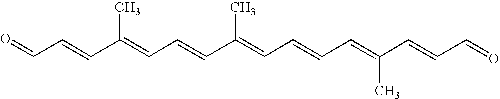 |
| diapo-6,12′-lycopendial, (6,12′) | 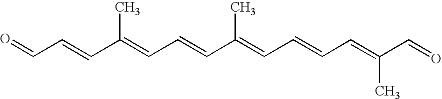 |
| diapo-10,8′-lycopendial (10,8′) | 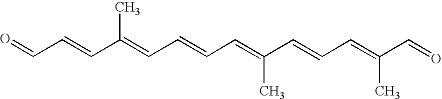 |
| diapo-8,12′-lycopendial (8,12′) | 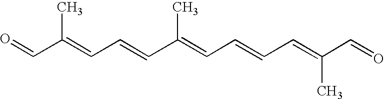 |
| 2,4,6-octatrienedial,2-(hydroxymethyl)-7-methyl-(E,E,E)- | 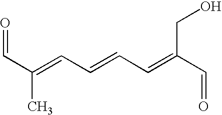 |
| 5-Cis-4,4′-7,7′,8,8′,11,11′,12,12′,15,15′-decahydro-diapo-ψ,ψ-carotenedial | 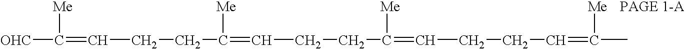 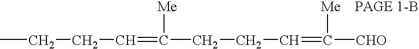 |
| 7,7′,8,8′,11,11′,12,12′,15,15′-Decahydro-4,4′-diapo-ψ,ψ-carotenedial | 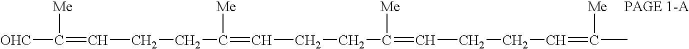 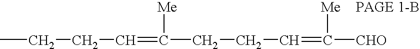 |
| 7,7′,8,8′,11,11′,12,12′,15,15′-Decahydro-6,6′-diapo-ψ,ψ-carotenedial | 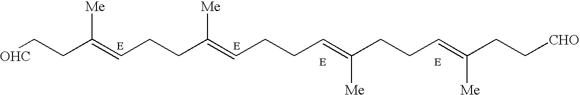 |

In one embodiment, the carotenoid oxidation product is other than 2,7,11-trimethyl-tetradecahexaene-1,14-dial (also designated herein diapo-6,12′-lycopenedial).

Other preferred lycopene oxidation products that are encompassed by the present invention include aldehyde derivatives (referred to herein interchangeably as apo-lycopenals or apo-carotenals). Such dialdehyde derivatives are described, for example, by Ferreira et al. (8), Kim et al. (9) and Caris-Veyrat et al. (10). Examples of these aldehyde derivatives include but are not limited to apo-6′-lycopenal; apo-8′-lycopenal; apo-10′-lycopenal; apo-12′-lycopenal; apo-1,4′-lycopenal; apo-15-lycopenal (acycloretinal); apo-11-lycopenal (3,7,11-trimethyl-2,4,6,10-dodecatetraen-1-al); apo-7-lycopenal (3,7-dimethyl-2,6-octadien-1-al); 3,4-dehydro-5,6-dihydro-15,15′-apo-lycopenal; and the like. The structures of these derivatives are shown in Table 2 below.

TABLE 2
CHEMICAL STRUCTURE OF APO-LYCOPENALS

| Name | Chemical Structure |
|---|---|
| apo-6′-lycopenal | 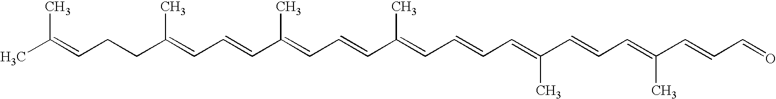 |
| apo-8′-lycopenal | 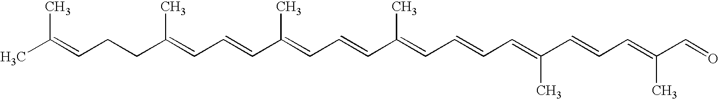 |
| apo-10′-lycopenal | 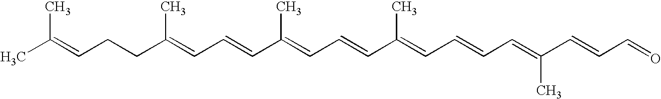 |
| apo-12′-lycopenal | 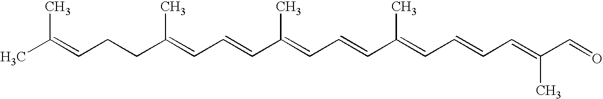 |
| apo-14′-lycopenal | 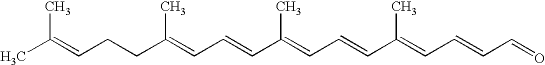 |
| apo-15-lycopenal(acycloretinal) | 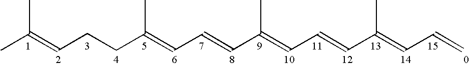 |
| apo-11-lycopenal | 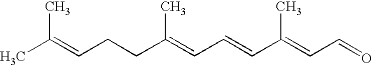 |
| apo-7-lycopenal | 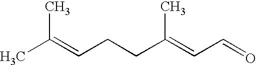 |
| 3,4-dehydro-5,6-dihydro-15,15′-apo-lycopenal | 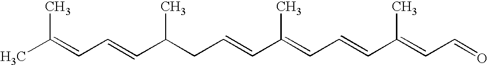 |

Further preferred examples of lycopene oxidation products that can be used in the methods and compositions of the present invention include ketone derivatives (referred to herein interchangeably as apo-lycopenones or apo-carotenones). Examples of these ketone derivatives include but are not limited to apo-13-lycopenone (6,10,14-trimethyl-3,5,7,9,13-pentadecapentaen-2-one); 3-keto-apo-13-lycopenone; apo-9-lycopenone (6,10-dimethyl-3,5,9-undecatrien-2-one); apo-5-lycopenone (2-methyl-2-hepten-6-one) (16), and the like. The structures of these derivatives are shown in Table 3 below.

TABLE 3
CHEMICAL STRUCTURE OF APO-LYCOPENONES

| Name | Chemical Structure |
|---|---|
| apo-13-lycopenone | 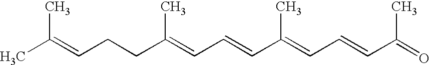 |
| 3-keto-apo-13-lycopenone | 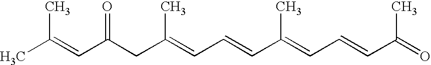 |
| apo-9-lycopenone | 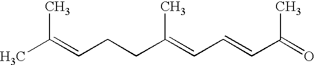 |
| apo-5-lycopenone | 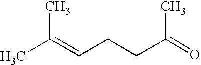 |

Further examples of lycopene oxidation products that can be used in the methods and compositions of the present invention include epoxide derivatives. Examples of such epoxide oxidation products include but are not limited to lycopene-5,6-5′,6′-diepoxide; lycopene 5,6-epoxide (2,8,10,12,14,16,18,20,22,24,26,30-dotriacontadodecaene,6,7-epoxy,2,6,10,14,19,23,27,31-octamethyl-(6CI))-CAS No. 51599-10-1); lycopene 1,2-1′,2′-diepoxide (CAS No. 76682-21-8); lycopene 1,2-epoxide (CAS No. 51599-09-8); and the like. The structures of these derivatives are shown in Table 4 below.

TABLE 4
CHEMICAL STRUCTURE OF EPOXIDE OXIDATION PRODUCTS

| Name | Chemical Structure |
|---|---|
| lycopene-5,6-5′,6′-diepoxide | 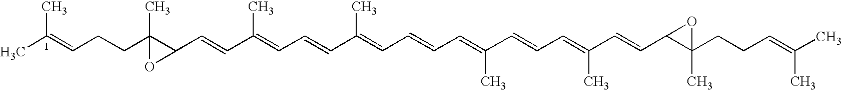 |
| lycopene5,6-epoxide | 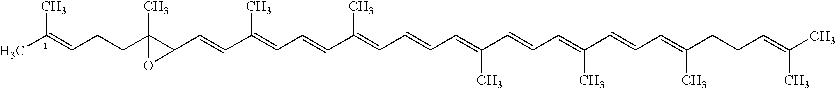 |
| lycopene1,2-1′,2′diepoxide | 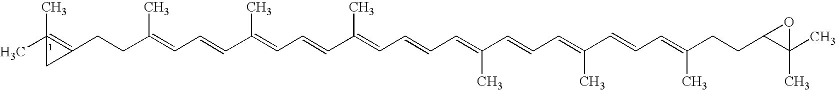 |
| lycopene1,2-epoxide | 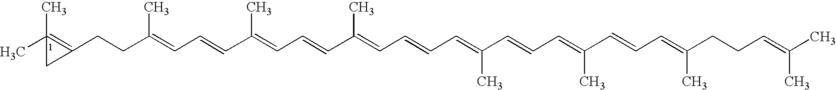 |

Further examples of lycopene oxidation products that can be used in the methods and compositions of the present invention include faranoxide derivatives. Examples of such furanoxide oxidation products include but are not limited to 3-keto-lycopene-5′8′-fluranoxide; lycopene-5,8-faranoxide Isomer (I); lycopene-5,8-furanoxide Isomer (II); 2-apo-5,8-lycopenal-fluranoxide as described in Ferreira et al (8); 1,5-epoxyiridanyl-lycopene (2-oxabicyclo[2.2.1]heptane, 7-(3,7,12,16,20,24-hexamethyl-1,3,5,7,9,11,13,15,17,19,23-pentacosaundecaenyl)-1,3,3-trimethyl-[1R[1α,4α,7S*(1E,3E,5E,7E,9E,11E,13E,15E, 17E,19E)]]-) (CAS No. 189620-82-4) (5); and the like. The structures of these derivatives are shown in Table 5 below.

TABLE 5
CHEMICAL STRUCTURE OF FURANOXIDE OXIDATION PRODUCTS

| Name | Chemical Structure |
|---|---|
| 3-keto-lycopene-5′8′-furanoxide | 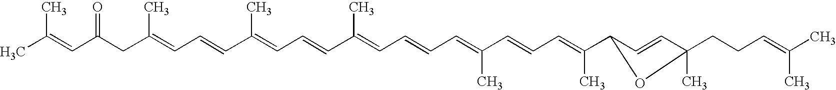 |
| lycopene-5,8-furanoxideIsomer (I) | 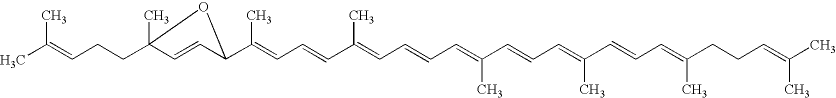 |
| lycopene-5,8-furanoxideIsomer (II) | 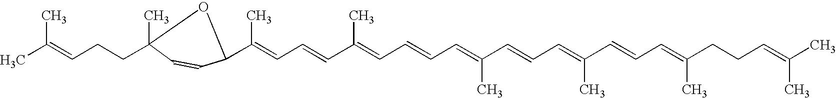 |
| 2-apo-5,8-lycopenal-furanoxide | 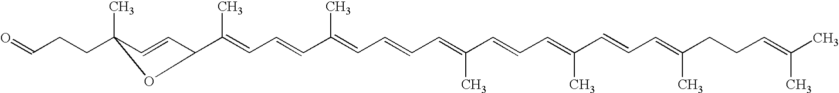 |
| 1,5-epoxy-iridanyl-lycopene | 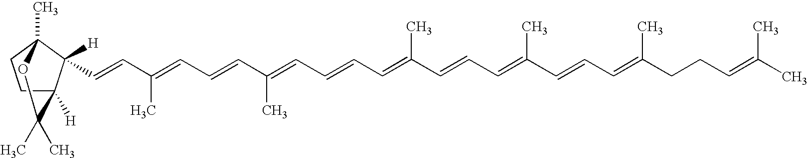 |

Other suitable lycopene oxidation products that can be used in the methods and compositions of the present invention are carboxylic acid derivatives including but not limited to acycloretinoic acid (9) and 6′-Apolycopenoic acid (CAS No. 22255-38-5). The structures of these compounds are shown in Table 6 below.

TABLE 6
CHEMICAL STRUCTURE OF CARBOXYLIC
ACID OXIDATION PRODUCTS

| Name | Chemical Structure |
|---|---|
| AcRA acyclo-retinoic acid | 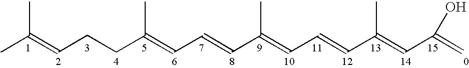 |
| 6′-Apolycopenoicacid | 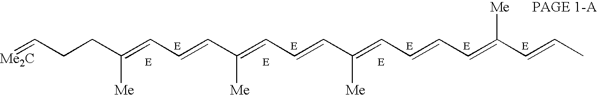 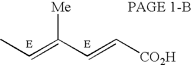 |

The lycopene oxidation products that can be used in the methods and compositions of the present invention can further include derivatives of any of the oxidation products described above. For example, acetal, ketal, acetylated, hydroxylated, and halogenated derivatives of any of the lycopene derivatives described above, are also included within the broad scope of the present invention. Also included are lycopene derivatives containing alkynic bonds or hydrogenated double bonds. Such compounds include but not limited to 15,15′-didehydro-(11-cis,11′-cis)-8,8′-diapo-ψ,ψ-carotenedial (CAS No. 97058-13-4); 15,15′-didehydro-11-cis-8,8′-diapo-ψ,ψ-carotenedial (CAS No. 96996-98-4); 15,15′-didehydro-8,8′-diapo-ψ,ψ-carotenedial (CAS No. 96948-31-1); 20,20′-dihydroxy-8,8′-diapo-ψ,ψ-carotenedial (CAS No. 56218-26-9); 20-hydroxy-8,8′-diapo-ψ,ψ-carotenedial (CAS No. 54795-87-8); 20-hydroxy-13-cis-8,8′-diapo-ψ,ψ-carotenedial (CAS No. 64474-13-1); 20,20′-bis(acetyloxy)-8,8′-diapo-ψ,ψ-carotenedial (CAS No. 56218-22-5); 20-(acetyloxy)-13-cis-8,8′-diapo-ψ,ψ-carotenedial (CAS No. 64474-12-0); 20-(acetyloxy)-8,8′-diapo-ψ,ψ-carotenedial (CAS No. 56218-21-4), 20,20′-dibromo-8,8′-diapo-ψ,ψ-carotenedial (CAS No. 56218-20-3); 20-Bromo-8,8′-diapo-ψ,ψ-carotenedial (CAS No. 56218-19-0); 6′-apolycopenal, dimethyl acetal (CAS No. 22255-37-4). The structures of these compounds are shown in Table 7 below.

TABLE 7

| Name | Chemical Structure |
|---|---|
| 15,15′-didehydro-,(11-cis,11′-cis)-8,8′-diapo-ψ,ψ-carotenedial | 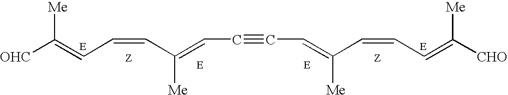 |
| 15,15′-didehydro-11-cis-8,8′-diapo-ψ,ψ-carotenedial | 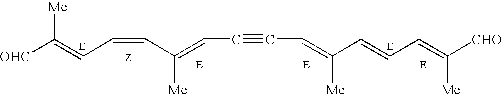 |
| 15,15′-didehydro-8,8′-diapo-ψ,ψ-carotenedial | 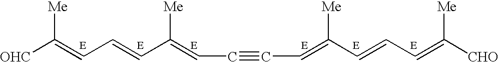 |
| 20,20′-dihydroxy-8,8-diapo-ψ,ψ-carotenedial | 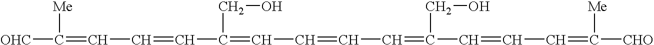 |
| 20-hydroxy-8,8′-diapo-ψ,ψ-carotenedial | 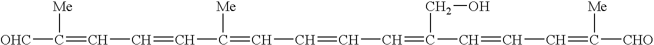 |
| 20-hydroxy-13-cis-8,8′-diapo-ψ,ψ-carotenedial | 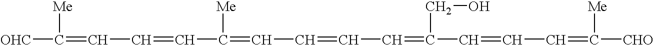 |
| 20,20′-bis(acetyloxy)-8,8′-diapo-ψ,ψ-carotenedial | 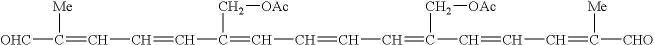 |
| 20-(acetyloxy)-13-cis-8,8′-diapo-ψ,ψ-carotenedial | 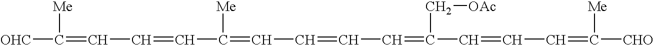 |
| 20-(acetyloxy)-8,8′-diapo-ψ,ψ-carotenedial. | 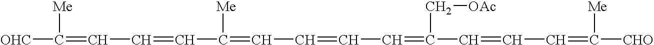 |
| 20,20′-dibromo-8,8′-diapo-ψ,ψ-carotenediall | 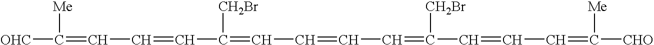 |
| 20-Bromo-8,8′-diapo-ψ,ψ-carotenedial | 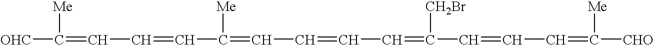 |
| 6′-Apolycopena1,dimethylacetal | 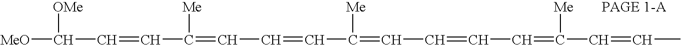 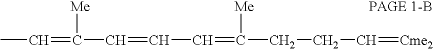 |

The present invention further contemplates the use of any other lycopene oxidation product not included in one of the aforementioned categories. Non-limiting examples of additional oxidation products include but are not limited to (E,E,E)-4-methyl-8-oxo-2,4,6-nonatrienal (34); (2S*,5S*,6R*)-2,6-cyclolycopene-1-methoxy-5-ol (41); (2S*,5S*,6R*),1-16-didehydro-2,6-cyclolycopene-5-ol (41); 2,6-cyclolycopene-1,5-diol (also called 1,5-dihydroxyiridanyl-lycopene) (27, 39); lycopene-1,2-dihydro-1-hydroxy-(7CI) (CAS No. 105-92-0); 1,1′,2,2′,-tetrahydro-1,1′-dihydroxylycopene (CAS No. 4212-57-1); 5,6-dihydro-5,6-dihydroxylycopene (CAS No. 66803-17-6), and the like. The structures of these compounds are shown in Table 8 below.

TABLE 8

| Name | Chemical Structure |
|---|---|
| (E,E,E)-4-methyl-8-oxo-2,4,6-nonatrienal | 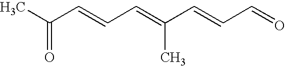 |
| (2S*,5S*,6R*)-2,6-cyclolycopene-1-methoxy-5-ol | 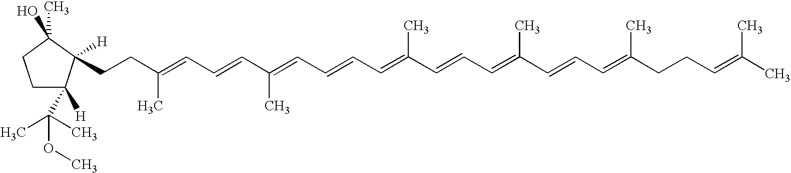 |
| (2S*,5S*,6R*),1,16-didehydro-2,6-cyclolycopene-5-ol | 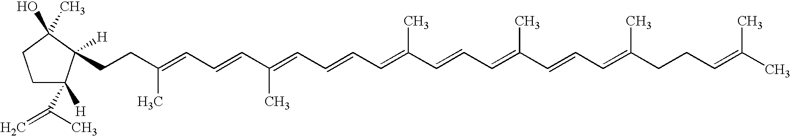 |
| 2,6-cyclolycopene-1,5-diol, alsodesignated 1,5-dihydroxy-iridanyl-lycopene | 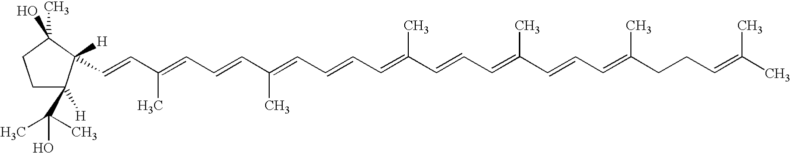 |
| lycopene-1,2-dihydro-1-hydroxy | 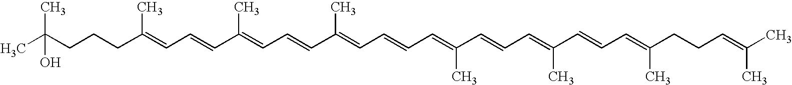 |
| 1,1′,2,2′-tetrahydro-1,1′-dihydroxy-lycopene | 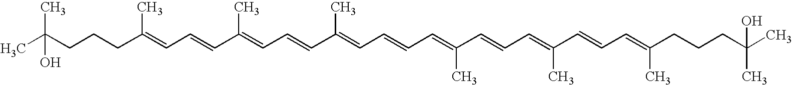 |
| 5,6-dihydro-5,6-dihydroxy-lycopene | 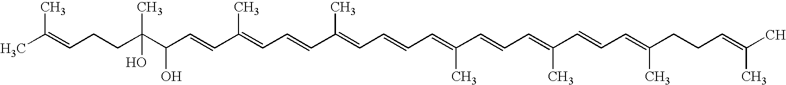 |

Also, by chemical reactions one can convert alkenes into gamma-lactones. diols and Alpha-hydroxy ketones, as well known to persons of skill in the art. The use of any of these derivatives is also contemplated within the broad scope of the present invention. Acetal and ketal derivatives are also contemplated, as described for example in US 2003/0158427, the contents of which are incorporated by reference in their entirety as if fully set forth herein.

In addition, any mixtures of one or more of the lycopene oxidation products described hereinabove, can also be used in the methods and compositions of the present invention. As contemplated herein, such mixtures include mixtures of synthetic lycopene derivatives, mixtures formed by oxidation of lycopene using any of the enzymatic and non-enzymatic procedures known in the art, as well as mixtures obtained by oxidation of lycopene in carotenal (6CI); 8′-Apo-β-carotenal, all-trans-(8CI); β-apo-Carotenal; 2,4,6,8,10,12,14,16-Heptadecaoctaenal, 2,6,11,15-tetramethyl-17-(2,6,6-trimethyl-1-cyclohexen-1-yl)-, (all-E)-; 8′-Apo-β,ψ-caroten-8′-al; 8′-Apo-β-caroten-8′-al; 8′-Apo-caroten-8′-al; 8′-apo-β-Caroten-8′-al; C Orange 16; C.I. 40820; C.I. Food Orange 6; E 160e; all-trans-β-Apo-8′-carotenal); 5) 4-hydroxy-beta-apo-13-carotenone (also named 3,5,7-Octatrien-2-one, 8-(3-hydroxy-2,6,6-trimethyl-1-cyclohexen-1-yl)-6-methyl-, (3E,5E,7E)-(9CI)); 6) 12′-Apo-β,ψ-carotenal, 13′-cis (also named 13′-cis-β-Apo-12′-carotenal); 7) β,β-Carotene-5,6-epoxide (also named β,β-Carotene, 5,6-epoxy-5,6-dihydro-(9CI); β-Carotene, 5,6-epoxy-5,6-dihydro-(7CI); β-Carotene, 5,6-epoxy-5,6-dihydro-, all-trans-(8CI); 7-Oxabicyclo[4.1.0]heptane, β-Carotene 5,6-epoxide; β-Carotene 5,6-monoepoxide; β-Carotene monoepoxide; 5,6-Dihydro-β,β-carotene 5,6-epoxide; 5,6-Epoxy-β-carotene; 5,6-Epoxy-5,6-dihydro-β,β-carotene; 5,6-Monoepoxy-β-carotene); and 8) 5,8-Epoxy-5,8-dihydro-β,β-carotene (also named β,β-Carotene, 5,8-epoxy-5,8-dihydro-(9CI); β-Carotene, 5,8-epoxy-5,8-dihydro-, all-trans-(8CI); β-Carotene 5,8-epoxide; 5,8-Epoxy-5,8-dihydro-β,β-carotene. The chemical structures of the above compounds are listed in Table 9 below.

TABLE 9
CHEMICAL STRUCTURE OF BETA-CAROTENE
OXIDATION PRODUCTS

| | Chemical | Chemical | |
|---|---|---|---|
| CAS No. | Name | Formula | Chemical Structure |
| 79-77-6 | (E)-β-Ionone | C₁₃H₂₀O | 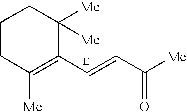 |
| 6985-27-9 | β-apo-14′-Carotenal | C₂₂H₃₀O | 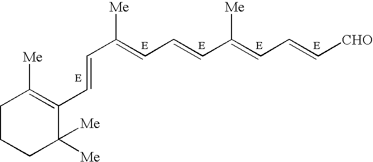 |
| 640-49-3 | β-apo-10′-Carotenal | C₂₇H₃₆O | 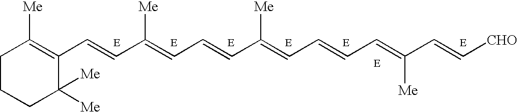 |
| 1107-26-2 | β-Apo-8′-carotenal | C₃₀H₄₀O | 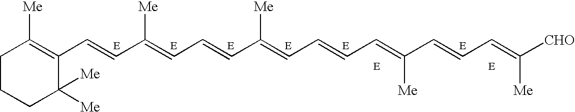 |
| 488834-48-6 | 4-hydroxy-beta-apo-13-carotenone | C₁₈H₂₆O₂ | 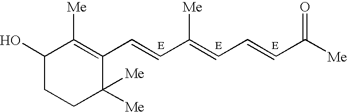 |
| 173937-56-9 | 12′-Apo-β,ψ-carotenal,13′-cis | C₂₅H₃₄O | 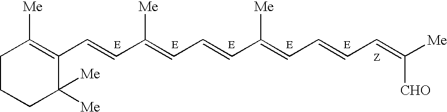 |
| 1923-89-3 | 3 β,β-Carotene-5,6-epoxide | C₄₀H₅₆O | 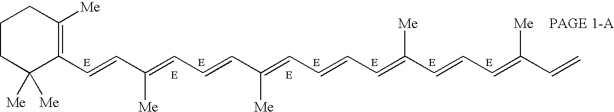 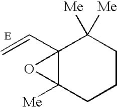 |
| 15678-54-3 | 5,8-Epoxy-5,8-dihydro-β,β-carotene | C₄₀H₅₆O | 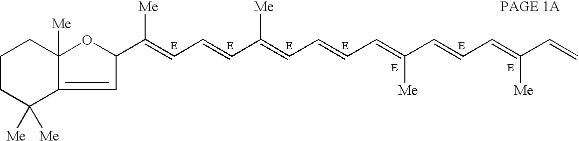 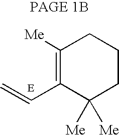 |

Other suitable β-carotene oxidation products encompassed within the broad scope of the present invention include but are not limited to beta-apo-13-carotenone; retinal; beta-apo-12′-carotenal; 15,15′-epoxy-beta,beta-carotene; and 11,15′-cyclo-12,15-epoxy-11,12,15,15′-tetrahydro-beta-carotene.

It is apparent to a person of skill in the art that list is in no way exhaustive, and should be used as representative examples and not limiting to other β-carotene oxidation products that can be used in the methods and compositions of the present invention. Thus, any one or more of the oxidation products described hereinabove with respect to lycopene, can also be applied to β-carotene. Thus, the present invention contemplates the use of β-carotene dialdehyde derivatives, aldehyde derivatives, ketone derivatives, epoxide derivatives, faranoxide derivatives, carboxylic acid derivatives, gamma-lactone derivatives, alpha-hydroxy ketone derivatives, diol derivatives, acetal derivatives, ketal derivatives, halogenated derivatives, acetylated derivatives, derivatives containing one or more alkynic bonds, hydrogenated derivatives in which one or more of the double bonds has been reduced, and the like. Combinations of one or more of these functional groups are also contemplated. Compounds containing more than one functional group are also contemplated. Such oxidation products can be synthetically prepared in accordance with any one of the methods described above, or these products can be formed in-vivo, in biological systems, as described above.

### C. Other Carotenoid Derivatives

It should be apparent to a person of skill in the art, that the present invention is not limited to the carotenoid derivatives described above. Rather, the present invention contemplates the use of any oxidation product of any carotenoid, whether synthetic or natural, that is found in any fruits and vegetables. As shown below, the difference in the structure of lycopene and β-carotene lies beyond the 8-position of each side of the symmetric molecule. Thus, all compounds that are derivatives from the 8 position to position 15 can be obtained from every carotenoid that has the same conjugated double bond system between the two symmetric 8 positions. This includes all carotenoids with beta-ionone ring, including but not limited to α- and β-carotene, lutein, zeaxanthin, phytoene, phytofluene, α- and β-cryptoxanthin, canthaxanthin and astaxanthin. The last two (canthaxanthin and astaxanthin) are usually not found in blood because of low consumption and can be commercially important.

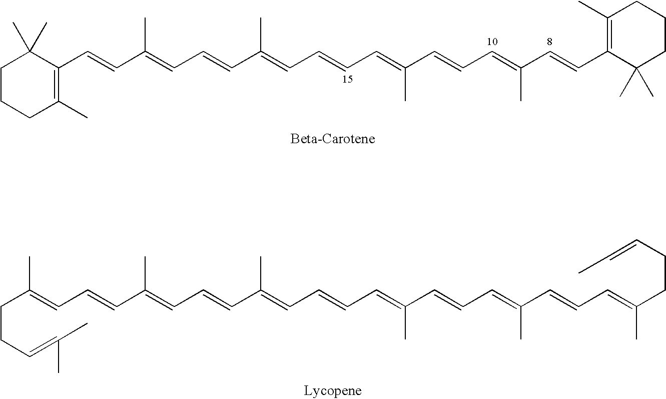

Oxidation of these compounds (not including dehydrogenation) should give rise to hydrogenated analogues of the oxidation products of lycopene shown above (for example cleavage of the central bond). Thus, the present invention also contemplates the use of any one or more of synthetic or natural carotenoid derivatives which are putative oxidation products of these carotenoids, including but not limited to dialdehyde derivatives, aldehyde derivatives, ketone derivatives, epoxide derivatives, furanoxide derivatives, carboxylic acid derivatives, gamma-lactone derivatives, alpha-hydroxy ketone derivatives, diol derivatives, acetal derivatives, ketal derivatives, halogenated derivatives, acetylated derivatives, derivatives containing one or more alkynic bonds, hydrogenated derivatives in which one or more of the double bonds has been reduced, and the like. Combinations of one or more of these functional groups are also contemplated. Compounds containing more than one functional group are also contemplated. These derivatives can be obtained in a similar manner to the lycopene oxidation products, as described above.

The invention includes pharmaceutically acceptable salts of the carotenoid oxidation derivatives of the present invention. Pharmaceutically acceptable salts can be prepared by treatment with inorganic bases, for example, sodium hydroxide or inorganic/organic acids such as hydrochloric acid, citric acids and the like. The term “pharmaceutically acceptable salts” refers to salts prepared from pharmaceutically acceptable non-toxic bases or acids including inorganic or organic bases and inorganic or organic acids. It is to be understood that, as used herein, references to the carotenoid oxidation products of the present invention are meant to also include the pharmaceutically acceptable salts thereof.

The invention also includes hydrates of the carotenoid oxidation products of the present invention. The term “hydrate” includes but is not limited to hemihydrate, monohydrate, dihydrate, trihydrate and the like. The invention also includes all crystalline polymorphic forms and all amorphous forms of the carotenoid oxidation products of the present invention

### Mechanism of Action

Without wishing to be bound by any particular mechanism and theory, it believed that the carotenoid oxidation products may exert their powerful effects by inducing antioxidant response element (ARE), which in turn induces the expression of phase II detoxification enzymes. These enzymes detoxify many harmful substances by converting them into hydrophilic metabolites that can be excreted readily from the body. Examples of phase II enzymes, are NAD(P)H:quinone oxidoreductase (NQO1) and g glutamylcysteine synthetase (GCS). The major ARE-activating transcription factor Nrf2 plays a central role in the induction of antioxidant and detoxifying genes.

In one non-limiting embodiment described herein for purpose of illustration, the carotenoid oxidation product increases the activity of ARE by forming keap 1 adducts with the carotenoid oxidation product. This may cause in turn its dissociation from Nrf2 and the activation of the latter. Keap1 is cysteine-rich cytoplasmic protein that negatively regulates the activation of Nrf2. The central domain of Keap1 is the most cysteine-rich domain and is required for cytoplasmic sequestration and inhibition of Nrf2. Dissociation of Nrf2 from Keap1 represents a regulatory step that allows Nrf2 to translocate to the nucleus and activate transcription of ARE-dependent genes which are important for cancer prevention. The dissociation of Nrf2 from Keap1-Nrf2 may be regulated by the redox status of these specific cysteine residues. Adducts of specific aldehydes were observed in LC-MS-MS analyses of purified human Keap1. The formation of these adducts was coincident with. Nrf2 stabilization, nuclear Nrf2 translocation and ARE dependent gene activation.

The term “induce” and variants thereof as used herein, has its commonly known meaning of increase or elevate. The term “induce an antioxidant response element” refers to inducing the level of the ARE, the activity of the ARE, the expression of ARE, or any combination thereof. For example, the applicants of the present invention have demonstrated that the carotenoid oxidation products increase the activity of ARE in several cancer cell lines.

The term “activity” as used herein refers to enzymatic activity. The term “level” as used herein refers to protein level, gene (DNA) level, RNA level (e.g., m-RNA), or any combination thereof. The term “expression” as used herein refers to gene expression or protein expression.

Thus, in one embodiment, the present invention provides in vitro methods for inducing an antioxidant response element, by contacting the antioxidant response element with an effective amount of a carotenoid oxidation product.

In another aspect, the present invention provides in vivo methods for inducing an antioxidant response element in a cell, by contacting a cell containing the antioxidant response element with a carotenoid oxidation product. The cell can be a non-malignant (non-cancer) cell, a pre-malignant (re-cancer) cell, or a malignant (cancer) cell.

In yet another embodiment, the present invention provides in vivo methods of inducing a phase II detoxification enzyme in a cell containing such phase II detoxification enzyme, by contacting the cell with a carotenoid oxidation product, in an amount effective to induce an antioxidant response element in the cell, thereby inducing the phase II detoxification enzyme. The cell can be a non-malignant (non-cancer) cell, a pre-malignant (pre-cancer) cell, or a malignant (cancer) cell.

It is apparent to a person of skill in the art that the present invention is not limited to the aforementioned mechanism of action and that compounds of the present invention may act on other cellular targets to achieve the anticancer effects described herein.

### Therapeutic Use

As described herein, the carotenoid oxidation products of the present invention are potent chemopreventive and chemotherapeutic agents that are capable of inhibiting cancer cell proliferation in a wide variety of cancer cells. The present invention thus provides powerful methods to the chemoprevention and treatment of cancer that have not been previously described.

Thus, in one aspect, the present invention relates to a method for the prevention and treatment of cancer by administering a pharmaceutical composition comprising a carotenoid oxidation product.

In another embodiment, the present invention provides a method of preventing the onset of cancer in a subject, comprising the step of administering to the subject a pharmaceutical composition comprising a carotenoid oxidation product, in an amount effective to prevent the onset of cancer in the subject.

In another embodiment, the present invention provides a method of inhibiting cancer cell proliferation in a subject, comprising the step of administering to the subject a pharmaceutical composition comprising a carotenoid oxidation product, in an amount effective to inhibit cancer cell proliferation in the subject.

In yet another embodiment, the present invention provides a method of delaying the progression of cancer in a subject, comprising the step of administering to the subject a pharmaceutical composition comprising a carotenoid oxidation product, in an amount effective to delay the progression of cancer in the subject.

In yet another embodiment, the present invention provides a method of preventing the recurrence of cancer in a subject, comprising the step of administering to the subject a pharmaceutical composition comprising a carotenoid oxidation product, in an amount effective to prevent the recurrence of cancer in the subject.

As demonstrated herein, the carotenoid derivatives of the present invention inhibit cancer cell proliferation which is important for delaying cancer progression. Inhibition of cell proliferation is important for the treatment of cancer as it slowed down cancer progression. Thus, in another embodiment, the present invention provides a method of treating cancer in a subject, comprising the step of administering to the subject a pharmaceutical composition comprising a carotenoid oxidation product, in an amount effective to treat cancer in the subject.

In one embodiment, the carotenoid oxidation products are more active, for example at least 10% more active, preferably at least 50% more active, and more preferably at least two fold more active, as compared with the parent carotenoid. The oxidation product may even display up to ten fold higher activity as compared with the parent carotenoid product. By “more active” it is meant, for example, that the oxidation product is generally more effective as an anti-cancer agent, as compared with the parent carotenoid molecule. For example, the oxidation product can induce ARE, inducing a phase II detoxification enzyme, inhibit cancer cell proliferation, prevent the onset of cancer and delay the progression of cancer, at a lower dose and with greater potency, as compared with the parent carotenoid molecule.

Further, the in-vivo activities of the oxidation products can differ from their in-vitro activities. Generally, the oxidation products are more polar than their parent counterparts, which may affect certain properties such as solubility, dissolution and bioavailability. As such, the oxidation products may be more potent chemotherapeutic agents compared with the corresponding parent molecule.

As used herein, the term “treating” includes preventative as well as disorder remitative treatment. As used herein, the term “inhibiting” used herein interchangeably with the terms “reducing” or “suppressing”, has its commonly understood meaning of lessening or decreasing. As used herein, the term “progression” means increasing in scope or severity, advancing, growing or becoming worse. As used herein, the term “recurrence” means the return of a disease after a remission.

As used herein, the term “administering” refers to bringing in contact with a carotenoid oxidation product of the present invention. Administration can be accomplished to cells or tissue cultures, or to living organisms, for example humans. In one embodiment, the present invention encompasses administering the compounds of the present invention to a human subject.

The terms “cancer” or “malignancy”, used herein interchangeably in the context of the present invention, include all types of neoplasm whether in the form of solid or non-solid tumors, from all origins, and includes both malignant solid or non-solid tumors as well as their metastasis. In particular this term refers to: carcinoma, sarcoma, adenoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell and non-small cell lung carcinoma, bladder carcinoma, epithelial carcinoma, glioma, astrocyoma, medulloblastoma, craniopharyngioma, ependynoma, pinealoma, retinoblastoma, multiple myeloma, rectal carcinoma, cancer of the thyroid, head and neck cancer, brain cancer, cancer of the peripherial nervous system, cancer of the central nervous system, neuroblastoma, cancer of the edometrium, myeloid lymphoma, leukemia, lymphoma, lymphoproliferative diseases, acute myelocytic leukemia, chronic leukemia, Hodgkin's lymphoma, non-Hodgkin's lymphoma, liver cancer as well as metastasis of all the above. More preferably, the cancer is selected from the group consisting of prostate cancer, liver cancer and breast cancer.

In other embodiments of the present invention, the carotenoid oxidation products can be administered in conjunction with one or more traditional chemotherapeutic agents or chemopreventive agents. The combination of a carotenoid derivative and the traditional drug may allow administration of a lesser quantity of the traditional drug, and thus the side effects experienced by the subject may be significantly lower, while a sufficient chemotherapeutic effect or chemopreventive effect is nevertheless achieved.

The carotenoid oxidation products of the present invention have been shown to inhibit proliferation of several cancer cell lines. Therefore, in another aspect, the invention also provides a method of inhibiting cancer cell proliferation, by contacting a cancer cell with a carotenoid oxidation product, in an amount effective to inhibit proliferation of the cancer cell.

As used through this specification and the appended claims, the singular forms “a”, “an” and “the” include the plural unless the context clearly dictates otherwise. Thus, for example, reference to “a carotenoid oxidation product” includes mixtures of such compounds, reference to “an antioxidant response element”, or “phase II enzyme” includes reference to respective mixtures of such molecules, reference to “the formulation” or “the method” includes one or more formulations, methods and/or steps of the type described herein and/or which will become apparent to those persons skilled in the art upon reading this disclosure.

### Pharmaceutical Compositions

Although the carotenoid oxidation products of the present invention can be administered alone, it is contemplated that these compounds will be administered in a pharmaceutical composition containing the carotenoid oxidation product together with a pharmaceutically acceptable carrier or excipient.

The pharmaceutical compositions of the present invention can be formulated for administration by a variety of routes including oral, rectal, transdermal, subcutaneous, intravenous, intramuscular, and intranasal. Such compositions are prepared in a manner well known in the pharmaceutical art and comprise as an active ingredient at least one carotenoid oxidation product as described hereinabove, together with a pharmaceutically acceptable excipient or a carrier. During the preparation of the pharmaceutical compositions according to the present invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

Some examples of suitable excipients include but are not limited to lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methylcellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 0.1 to about 500 mg of the active compound. The term “unit dosage forms” refers to physically discrete units suitable as unitary dosages for human subjects and other mammals, each unit containing a predetermined quantity of the active compound calculated to produce the desired therapeutic effect, in association with a suitable pharmaceutical excipient.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation is then subdivided into unit dosage forms of the type described above.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials include a number of polymeric acids and mixtures of polymeric acids with materials such as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXPERIMENTAL DETAILS SECTION

### Materials and Methods

### Materials

Putative lycopene oxidation products were synthesized by Dr. Hansgeorg Ernst from BASF. Crystalline lycopene purified from tomato extract (>97% pure when prepared) was a gift from LycoRed Natural Products Industries (Beer-Sheva, Israel). Tetrahydrofuran containing 0.025% butylated hydroxytoluene as an antioxidant was purchased from Aldrich (Milwaukee, Wis.). DMEM, MEM-Eagle medium, FCS, charcoal stripped, delipidated FCS, and Ca²⁺/Mg²⁺-free PBS were purchased from Biological Industries (Beth Haemek, Israel). DMSO and tertbutylhydroquinone (tBHQ) were purchased from Sigma. Acycloretinoic acid (acRA) was obtained from Dr. Hansgeorg Ernst from BASF, and all-trans retinoic acid (atRA) was obtained from Sigma.

Lycopene, Synthetic Lycopene Derivatives and tBHQ Solutions

Lycopene and synthetic lycopene derivatives (putative lycopene oxidation products) were dissolved in tetrahydrofuran and solubilized in cell culture medium as described previously (8). tBHQ was dissolved in DMSO. The final concentrations of tetrahydrofuran and DMSO in the cell culture media were 0.5% and 0.1%, respectively. The vehicles did not affect the parameters measured in the presented experiments. All procedures were done under reduced lighting.

### Reporter Constructs and Expression Vectors

ARE reporter constructs were provided by Dr. J. J. Gipp (University of Wisconsin Medical School, Madison, Wis.; Ref 18). NQO1hARE-tk-luc and GCShARE4-tk-luc contain sequences of the active response elements from the promoters of human NQO1 and GCS heavy subunit, respectively. GCShARE4m-tk-luc, a non-active mutant form of the latter construct, contains a single base mutation in the relevant sequence.

### Cell Culture

MCF-7, human mammary cancer cells were purchased from American Type Culture Collection (Rockwell, Md.). MCF-7 cells were grown in DMEM containing penicillin (100 units/mL), streptomycin (0.1 mg/mL), nystatin (12.5 Ag/mL), 0.6 μg/mL insulin, and 10% FCS. T47D, human mammary cancer cell line, were provided by Dr. Yafa Keidar (Tel-Aviv University, Israel) and were grown in DMEM medium. LNCaP human prostate cancer cells were purchased from American Type Culture Collection (Rockwell, Md.) and were grown on RPMI medium. The medium contained penicillin (100 U/ml), streptomycin (0.1 mg/ml) nystatin (12.5 μg/ml), and 10% fetal calf serum (FCS), and 6 μg/ml insulin. Prior to each experiment, the cells were depleted of steroid hormones and maintained for 3-5 days in phenol red-free DMEM supplemented with 10% DCC FCS.

### Transient Transfection and Reporter Gene Assay

Cell transfection and reporter gene assays were generally carried out as follows. Some variations may be adopted for different cell lines: Cells were transfected using TFx-50 reagent (Promega, Madison, Wis.) with ca. 0.07 μg of DNA containing 0.05 μg of reporter plasmid and 0.02 μg of Renilla luciferase expression vector (P-RL-null vector, Promega) as an internal standard. For this purpose, cells were seeded in 24-well plates (100,000 cells per well). After 1 day, cells were rinsed twice with the appropriate culture medium without serum, followed by the addition of 0.2 mL of medium containing DNA and TFx-50 reagent at a charge ratio of 1:3. The cells were then incubated for 30 minutes at 37° C. in 95% air/5% CO₂. Four hundred microliters of medium containing 3% charcoal-stripped delipidated FCS were added and incubation continued for 8 hours. This was then replaced by medium supplemented with 3% delipidated FCS and the test compounds and cells were incubated for another 16 hours. Cell extracts were prepared for luciferase reporter assay (Dual Luciferase Reporter Assay System, Promega) according to the manufacturer's instructions.

### Real-Time PCR

Total RNA was extracted from cells with the RNeasy Mini Kit (Qiagen, Hilden, Germany) and cDNA was prepared as previously described (19). NQO1 and GCS mRNA were determined by quantitative real-time PCR and the results were normalized according to corresponding values of glyceraldehyde-3-phosphate dehydrogenase mRNA. The following primers were used:

NQO1 sense,
5V-CAACCACGAGCCCAGCCAAT A-3V;
NQO1 antisense,
5V-TTCAAAGCCGCTGCAGCAG-3V;
GCS sense,
5VACGAGGCTGAGTGTCCGTCT-3V;
GCS antisense,
5VTGGCGCTTGGTTTCCTC-3V;
glyceraldehyde-3-phosphate dehydrogenase sense,
5V-GTTCGACAGTCAGCCGCATC-3V;
glyceraldehyde-3-phosphate dehydrogenase anti-
sense,
5V-CGCCCAATACGACCAAATCC-3V.

cdNA samples (7 μL) were diluted 9-fold, mixed with the specific primers (0.2 mmol) and Thermo-Start master mix (ABgene, Surrey, United Kingdom). SYBR green I dye (Amresco, Cleveland) was then added to the reaction mixture. Reactions were carried out in the Rotor-Gene Real-Time PCR machine (Corbett-Research, Northlake, Australia). Standard cycling conditions for this instrument were, 15 minutes initial enzyme activation at 95° C., then 35 cycles as follows: 10 seconds at 95° C., 15 seconds at the annealing temperature (60° C. for NQO1 and GCS primers, 58° C. for glyceraldehyde-3-phosphate dehydrogenase primer) and 20 seconds at 72° C.

### Western Blotting

Cells were lysed as described previously (20) with modifications. Cell monolayers were washed twice with ice-cold PBS and then scraped into ice-cold lysis buffer A [50 mmol/L HEPES (pH 7.5), 150 mmol/L NaCl, 10% (v/v) glycerol, 1% (v/v) Triton X-100, 1.5 mmol/L MgCl₂, 1 mmol EGTA, 10 μg/mL aprotinin, 10 μg/mL leupeptin, 1 μmol/L phenylmethylsulfonyl fluoride, 2 mmol/L sodium orthovanadate, 10 mmoL sodium pyrophosphate, 50 mmol NaF, and 0.2 mmol/L DTT]. The lysates were incubated for 10 minutes on ice, and the cellular debris were cleared by centrifugation (20,000×g, 10 minutes, 4° C.). The protein content of the samples was determined by the Bradford method using a protein assay kit (Bio-Rad, Richmond. CA). Equal amounts of protein (30 μg) were separated by SDS PAGE and then transferred to a nitrocellulose membrane. Proteins were visualized using the SuperSignal West Pico chemiluminescence system (Pierce Chemical, Rockford, Ill.) after incubation overnight at 4° C. with the following primary antibodies: NQO1 (c-19, sc-16464), GCS (GSH1 N-20, sc-15085), from Santa Cruz Biotechnology, Inc. (Santa Cruz, Calif.). Protein abundance was quantitated by densitometric analysis using the ImageMaster VDS-CL imaging system (Amersham Pharmacia Biotech, Piscataway, N.J.).

### Cell Proliferation

MCF-7, LNCaP and T47D cells were seeded in 96-well plates. After one day, the medium was replaced with one containing the various materials. In each day of the experiment one plate was used for the thymidine incorporation assay. Thymidine incorporation was determined as follows: 2.5 μCi/well of [³H]thymidine (specific radioactivity 5 mCi/mmol) containing cold thymidine (100 μM) was added and the plate were incubated (37° C.) for 1 hr (MCF-7 cells) or for 3 hr (LNCaP, T47D cells). Nucleotide incorporation was stopped by adding unlabeled thymidine (0.5 μmol). The cells were then trypsinized and collected on a glass-fiber filter using a cell harvester (Inotech, Switzerland). Radioactivity was determined by a radioactive image analyzer (BAS 1000, Fuji, Japan).

### Example 1

### ARE Induction by Lycopene Derivatives

The effects of lycopene and several lycopene derivatives on antioxidant response element (ARE) induction were tested in all of the cancer cell lines mentioned above. The assay employed measures the transcriptional activity of the antioxidant response element which is activated by the Nrf2 transcription factor, and its activation by carotenoids and their derivatives. Parts of a promoter of the genes of interest were fused to a luciferase reporter gene as described in Materials and Methods. The constructs were transfected into the cells. Cells were incubated with carotenoids or derivatives and activation of transcription was measured.

The following synthetic lycopene derivatives were used: a) diapo-8,8′-lycopendial (8,8′); b) diapo-8′,12-lycopendial (8′,12); c) diapo-10,10′-lycopendial (10,10′); d) diapo-12,12′-lycopendial (12,12′); and e) diapo-8′,15-lycopendial (8′,15).

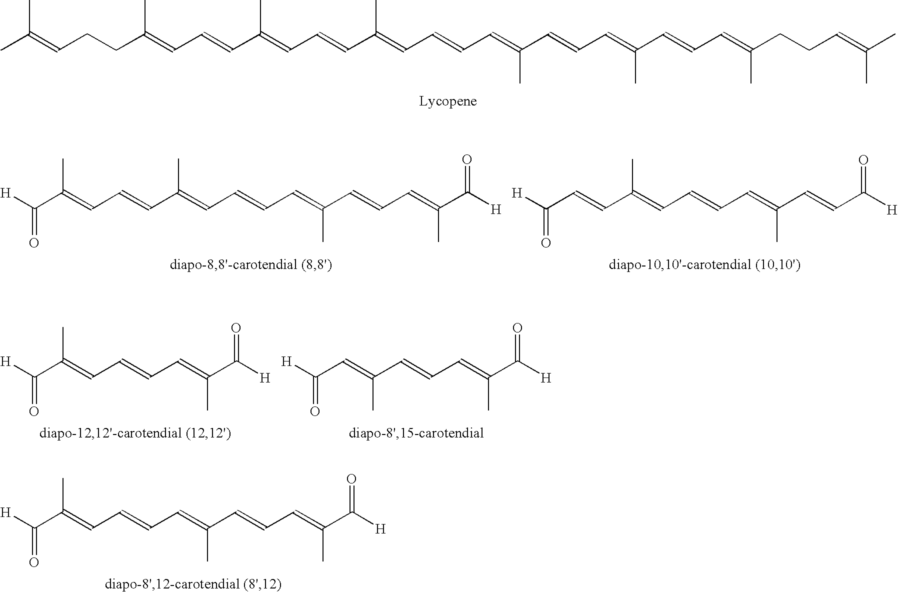

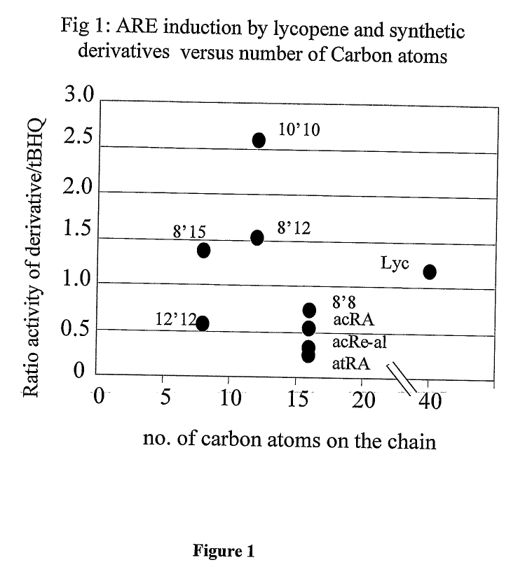

The results are depicted in FIG. 1 as the mean of 3-6 experiments. The standard errors of the mean (SEs) (not shown) were not higher than 10% of the mean. In order to decrease the variation between experiments, the results are expressed as the ratio of stimulation obtained with each specific derivative to that obtained with tBHQ (a well known positive control in this system).

As shown in FIG. 1, carotene dialdehydes stimulate the transcription of ARE reporter genes. Several of these derivatives (8′,15; 8′,12 and 10,10′) were more active than the parent molecule lycopene, and all derivatives were more active than other known retinoic acid derivatives such as all-trans retinoic acid (atRA), acycloretinoic acid (acRA) and acycloretinal (acRe-al).

The results also suggest that stimulation potency is related to the number of carbon atoms in the dialdehyde main chain. The 10′10 and 8′12 (12 carbon chain) derivatives are the most active, while the longest derivative (8′8-16 carbon chain) and the shortest one (12′12-8 carbon chain) were much less active. The 8′15 derivate (8 carbon chain) was also active, suggesting that other features of the compound, in addition the length of the carbon chain, are also important.

### Example 2

### Effect of Lycopene Derivatives on Mammary Cancer Cell Proliferation

The effect of lycopene, atRA and several lycopene derivatives ((a) diapo-8,8′-lycopendial; (b) diapo-8′,12-lycopendial; (c) diapo-10,10′-lycopendial; (d) diapo-12,12′-lycopendial; and (e) diapo-8′,15-lycopendial) on cell proliferation and ARE induction in two human breast cell lines (MCF-7 and T47D) was examined.

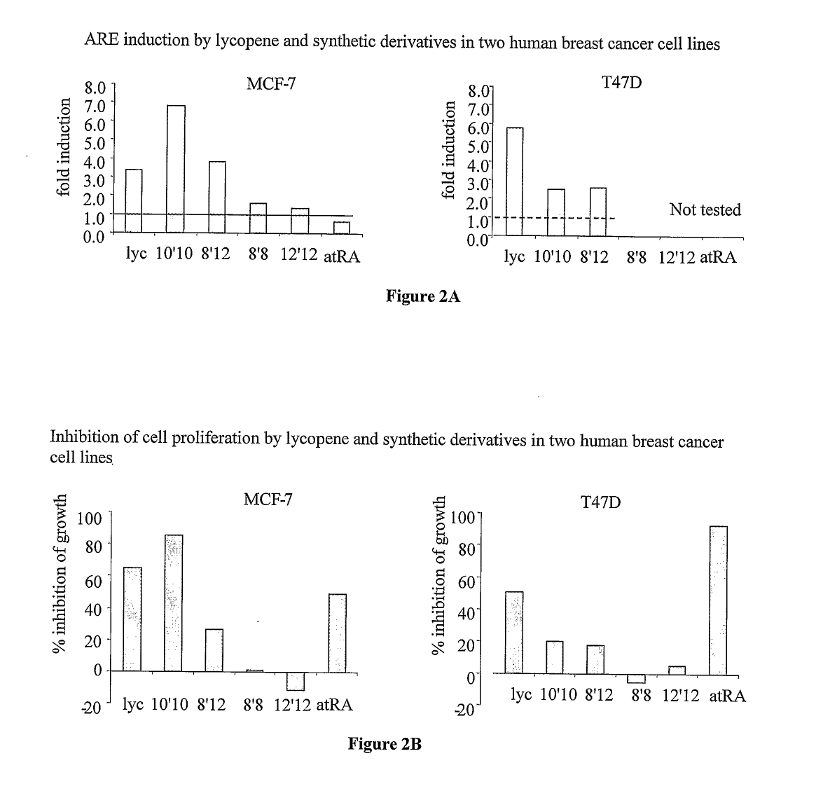

FIG. 2A shows the effect of several lycopene derivatives on ARE induction, and FIG. 2B shows the corresponding effect on cellular proliferation. As shown, mammary cancer cell proliferation was differentially inhibited by several synthetic dialdehyde lycopene derivatives.

The results further show that there is a significant correlation between the activation of ARE (FIG. 2A) and inhibition of cell proliferation (FIG. 2B). For example, the 10′10 derivative was more active than lycopene in both ARE induction and inhibition of cell proliferation, whereas the 8′8 derivative was less active in both assays.

### Example 3

### Effect of Lycopene Derivatives on Prostate Cancer Cell Proliferation

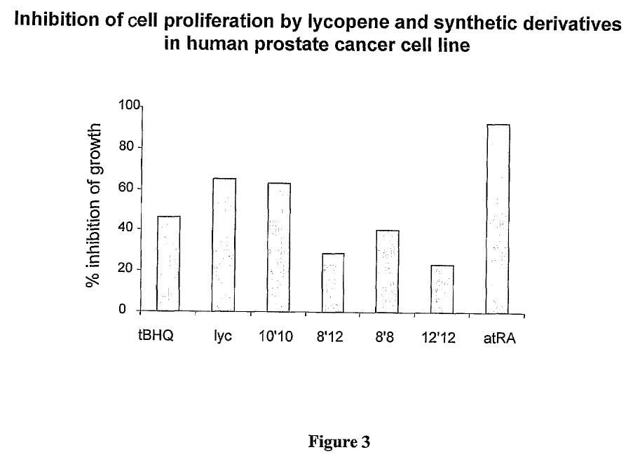

The effect of lycopene, atRA and several lycopene derivatives ((a) diapo-8,8′-lycopendial; (b) diapo-8′,12-lycopendial; (c) diapo-10,10′-lycopendial; (d) diapo-12,12′-lycopendial; and (e) diapo-8′,15-lycopendial) on cell proliferation and ARE induction in a LNCaP prostate cancer cell line was examined. As shown in FIG. 3, prostate cancer cell proliferation is also inhibited by these derivatives in a differential manner, demonstrating the ability of these derivatives to inhibit the proliferation of a wide range of cancer cells.

The results presented herein demonstrate that derivatives of lycopene, which are putative lycopene oxidation products, are potent inhibitors of cancer cell proliferation, and are thus useful agents for the treatment and prevention of cancer.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

### REFERENCES

1. van Poppel G. Carotenoids and cancer: an update with emphasis on human intervention studies. Eur J Cancer 1993; 29A: 1335-44.
2. Giovannucci E. Tomatoes, tomato-based products, lycopene, and cancer: review of the epidemiologic literature. J Natl Cancer Inst 1999; 91: 317-31.
3. Talalay P. Chemoprotection against cancer by induction of phase 2 enzymes. Biofactors 2000; 12: 5-11.
4. Ben-Dor A, Steiner M, Gheber L, Danilenko M, Dubi N, Linnewiel K, Zick A, Sharoni Y, and Levy J. Carotenoids activate the antioxidant response element transcription system. Mol. Cancer. Therp 2005; 4(1): 177-186.
5. Khachik F, Beecher G R, Smith J C. Lutein, lycopene and their oxidative metabolites in chemoprevention of cancer. J. Cell Biochem. 1995; 22:236-246 (Supplement).
6. King T J, Khachik F, Bortkiewicz H, Fukushima L H, Morioka S, Bertram J S. Metabolites of dietary carotenoids as potentioal cancer preventive agents. Pure & Appl Chem 1997; 69(10): 2135-40.
7. Bertram J S, King T, Fukishima L. Khachik F. Enhanced activity of an oxidation product of lycopene found in tomato products and human serum relevant to cancer prevention in: Sen, (Ed.). Antioxidant and Redox Regulation of Genes. Academic Press, New York, 2000, 409-24.
8. Ferreira A L, Yeum K J, Russell R M, Krinsky N I, Tang G. Enzymatic and oxidative metabolites of lycopene. J. Nutr. Biochem. 2003; 14: 531-540.
9. Kim S J, Nara E, Kobayashi H, Terao J, and Nagao A. Lipids, 2001; 36(2): 191-199.
10. Caris-Veyrat C, Schmid A, Carail M, and Bohm V. Cleavage products of lycopene produced by in vitro oxidations: characterization and mechanisms of formulation. J. Agric. Food Chem. 2003; 51: 7318-7325.
11. Araki, H., Shidoji, Y., Yamada, Y., Moriwaki, H., and Muto, Y. Retinoid agonist activities of synthetic geranyl geranoic acid derivatives, 1995; Biochem Biophys Res Commun. 209: 66-72.
12. Aust, O, Ale-Agha N, Zhang L, Wollersen H, Sies H, and Stahl W. Lycopene oxidation product enhances gap junctional communication. Food and Chem. Toxicol. 2003; 41: 1399-1407.
13. Zhang H., Kotake-Nara E, Ono H, and Nagao, A. A novel cleavage product formed by autoxidation of lycopene induces apoptosis in HL-60 cells. Free Radical Biol. & Med. 2003; 35(12):1653-1663.
14. Nara, E., Hayashi, H., Kotake, M., Miyashita, K., and Nagao, A. Acyclic carotenoids and their oxidation mixtures inhibit the growth of HL-60 human promyelocytic leukemia cells. Nut. & Cancer 2001; 39(2): 273-283.
15. Yokota, T, Etoh, H, Ukai H, Oshima S, Sakamoto, H. 1,5-dihydroxyiridanyl-lycopene in tomato puree. Biosci. Biotech Biochem. 1997; 61(3), 549-550.
16. Ukai, N, Lu, Y, Etoli, H, Yagi, A, Ina, K, Osima, S, Ojima, F, Sakamoto, H, and Ischiguro, Y. Biosci. Biotech Biochem. 1994; 58(9), 1718-1719.
17. Yokota, T, Etoh, H, Oshima, S, Hayakawa, K, and Ishiguro, Y, Biosci. Biotech Biochem. 2003; 67(12), 2644-2647.
18. Mulcahy R T, Wartman M A, Bailey H H, Gipp J J. Constitutive and β-naphthoflavone-induced expression of the human g-glutamylcysteine synthetase heavy subunit gene is regulated by a distal antioxidant response element/TRE sequence. J Biol Chem 1997; 272: 7445-54.
19. Basok A, Shnaider A, Man L, Chaimovitz C, Douvdevani A. CD40 is expressed on human peritoneal mesothelial cells and upregulates the production of interleukin-15 and RANTES. J Am Soc Nephrol 2001; 12: 695-702.
20. Watts C K, Brady A, Sarcevic B, DeFazio A, Musgrove E A, Sutherland R L. Antiestrogen inhibition of cell cycle progression in breast cancer cells in associated with inhibition of cyclin-dependent kinase activity and decreased retinoblastoma protein phosphorylation. Mol Endocrinol 1995; 9: 1804-13.

1-31. (canceled)

32. A method of preventing the onset of cancer, inhibiting cancer cell proliferation, delaying the progression of cancer or treating cancer in a subject, comprising the step of administering to said subject a pharmaceutical composition comprising an effective amount of a carotenoid oxidation product.

33. The method according to claim 32, wherein the carotenoid is selected from the group consisting of a tomato carotenoid, lycopene, α- and β-carotene, phytoene, phytofluene, lutein, zeaxanthin, α- and β-cryptoxanthin, canthaxanthin, astaxanthin, and combinations thereof.

34. The method according to claim 32, wherein the carotenoid oxidation product is a derivative selected from the group consisting of an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combinations thereof.

35. The method according to claim 32, wherein the carotenoid oxidation product is a lycopene oxidation product in the form of a dialdehyde derivative selected from the group consisting of diapo-8,8′-lycopendial (8,8′), diapo-8′,12-lycopendial (8′,12), diapo-10,10′-lycopendial (10,10′), diapo-12,12′-lycopendial (12,12′), diapo-8′,15-lycopendial (8′,15), and combinations thereof.

36. The method according to claim 32, wherein the carotenoid oxidation product induces an antioxidant response element (ARE) in said subject.

37. The method according to claim 32, wherein the cancer is selected from the group consisting of carcinoma, sarcoma, adenoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell and non-small cell lung carcinoma, bladder carcinoma, epithelial carcinoma, glioma, astrocyoma, medulloblastoma, craniopharyngioma, ependynoma, pinealoma, retinoblastoma, multiple myeloma, rectal carcinoma, cancer of the thyroid, head and neck cancer, brain cancer, cancer of the peripheral nervous system, cancer of the central nervous system, neuroblastoma, cancer of the edometrium, myeloid lymphoma, leukemia, lymphoma, lymphoproliferative diseases, acute myelocytic leukemia, chronic leukemia, Hodgkin's lymphoma, non-Hodgkin's lymphoma, liver cancer, and metastasis of all the above.

38. The method according to claim 32, wherein the subject is a human.

39. The method according to claim 32, wherein the carotenoid oxidation product is obtained from a naturally occurring carotenoid or is a synthetic compound.

40. A method of inhibiting cancer cell proliferation, comprising the step of contacting a cancer cell with a carotenoid oxidation product, in an amount effective to inhibit proliferation of said cancer cell.

41. The method according to claim 40, wherein the carotenoid is selected from the group consisting of a tomato carotenoid, lycopene, α- and β-carotene, phytoene, phytofluene, lutein, zeaxanthin, α- and β-cryptoxanthin, canthaxanthin, astaxanthin, and combinations thereof.

42. The method according to claim 40, wherein the carotenoid oxidation product is a derivative selected from the group consisting of an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combinations thereof.

43. The method according to claim 40, wherein the carotenoid oxidation product is a lycopene oxidation product in the form of a dialdehyde derivative selected from the group consisting of diapo-8,8′-lycopendial (8,8′), diapo-8′,12-lycopendial (8′,12), diapo-10,10′-lycopendial (10,10′), diapo-12,12′-lycopendial (12,12′), diapo-8′,15-lycopendial (8′,15), and combinations thereof.

44. The method according to claim 40, wherein the carotenoid oxidation product induces an antioxidant response element (ARE) in said cell.

45. The method according to claim 40, wherein the cancer is selected from the group consisting of carcinoma, sarcoma, adenoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell and non-small cell lung carcinoma, bladder carcinoma, epithelial carcinoma, glioma, astrocyoma, medulloblastoma, craniopharyngioma, ependynoma, pinealoma, retinoblastoma, multiple myeloma, rectal carcinoma, cancer of the thyroid, head and neck cancer, brain cancer, cancer of the peripheral nervous system, cancer of the central nervous system, neuroblastoma, cancer of the edometrium, myeloid lymphoma, leukemia, lymphoma, lymphoproliferative diseases, acute myelocytic leukemia, chronic leukemia, Hodgkin's lymphoma, non-Hodgkin's lymphoma liver cancer, and metastasis of all the above.

46. The method according to claim 40, wherein the cancer cell is a human cancer cell.

47. The method according to claim 40, wherein the carotenoid oxidation product is obtained from a naturally occurring carotenoid or is a synthetic compound.

48. A pharmaceutical composition comprising a carotenoid oxidation product, and a pharmaceutically acceptable carrier or excipient.

49. The pharmaceutical composition according to claim 48, wherein the carotenoid is selected from the group consisting of a tomato carotenoid, lycopene, α- and β-carotene, phytoene, phytofluene, lutein, zeaxanthin, α- and β-cryptoxanthin, canthaxanthin, astaxanthin, and combinations thereof.

50. The pharmaceutical composition according to claim 48, wherein the carotenoid oxidation product is a derivative selected from the group consisting of an aldehyde, a dialdehyde, a ketone, a carboxylic acid, an epoxide, a furanoxide, a gamma-lactone, an alpha-hydroxy ketone, a diol, an acetal, a ketal, a halogenated derivative, an acetylated derivative, a derivative containing one or more alkynic bonds, and combinations thereof.

51. The pharmaceutical composition according to claim 48, wherein the carotenoid oxidation product is a lycopene oxidation product in the form of a dialdehyde derivative selected from the group consisting of diapo-8,8′-lycopendial (8,8′), diapo-8′,12-lycopendial (8′,12), diapo-10,10′-lycopendial (10,10′), diapo-12,12′-lycopendial (12,12′), diapo-8′,15-lycopendial (8′,15), and combinations thereof.

52. The pharmaceutical composition according to claim 48, wherein the carotenoid oxidation product is obtained from a naturally occurring carotenoid or is a synthetic compound.
